(19) United States
(12) Patent Application Publication
(10) Pub. No.: US 2021/0163871 A1
(43) Pub. Date: Jun. 3, 2021

(54) INTEGRATED ALGAE HARVESTING AND GROWTH SYSTEMS AND METHODS RELATED THERETO

(71) Applicant: ExxonMobil Research and Engineering Company, Annandale, NJ (US)

(72) Inventors: Mark A Deimund, Jersey City, NJ (US); Patrick L Hanks, Bridgewater, NJ (US); Everett J O'Neal, Abury, NJ (US); Louis R Brown, La Jolla, CA (US)

(21) Appl. No.: 17/082,099

(22) Filed: Oct. 28, 2020

Related U.S. Application Data
(60) Provisional application No. 62/942,875, filed on Dec. 3, 2019.

(51) Int. Cl. C12M 1/26; B01D 46/54; C12N 1/12; C12M 1/00; B01F 3/04

(52) U.S. Cl. CPC C12M 33/14; B01D 46/543; B01F 3/04106; C12M 29/00; C12M 23/18; C12N 1/12

(57) ABSTRACT

Systems and methods for cultivating and harvesting algal biomass using cultivation vessels having integrated therewith one or more harvesting systems. Algae cells are cultivated in a cultivation system and concentrated algal biomass is collected using the integrated one or more harvesting systems. The integrated cultivating and harvesting systems reduce costs, energy, and time compared to traditional harvesting methods.

### CROSS-REFERENCE TO RELATED APPLICATIONS

This application claims the benefit of priority from U.S. Provisional Application No. 62/942,875 filed Dec. 3, 2019, which is herein incorporated by reference in its entirety.

### BACKGROUND OF THE INVENTION

Concerns about climate change, carbon dioxide (CO2) emissions, and depleting mineral oil and gas resources have led to widespread interest in the production of biofuels from algae, including microalgae. As compared to other plant-based feedstocks, algae have higher CO2 to fixation efficiencies and growth rates, and growing algae can efficiently utilize wastewater, biomass residue, and industrial gases as nutrient sources.

Algae are photoautotrophic organisms that can survive, grow, and reproduce with energy derived entirely from the sun through the process of photosynthesis. Photosynthesis is essentially a carbon recycling process through which inorganic CO2 combines with solar energy, other nutrients, and cellular biochemical processes to output gaseous oxygen and to synthesize carbohydrates and other compounds critical to the life of the algae.

To produce algal biomass in outdoor or large-area environments, algae is generally grown in a water slurry using one or more open pond systems, which are typically oval in shape (e.g., pill-shaped) and referred to as “raceway ponds.” The water slurry comprises selected nutrients, and the pond system circulates the algae in the water slurry to ensure adequate exposure to solar energy, thereby promoting the growth of algal biomass. Various processing methods separate the algal biomass and extract lipids therefrom for the production of fuel and other oil-based products. The remaining wastewater and biomass residue can be recycled or otherwise used in a variety of sustainable applications. For example, the wastewater can form some or all of a subsequent water slurry and the biomass residue can be used as animal feed.

Various processing methods exist for harvesting cultivated algal biomass to extract lipids therefrom for the production of fuel and other oil-based products. Moreover, harvesting cultivated algal biomass can be used to produce non-fuel or non-oil-based products, including nutraceuticals, pharmaceuticals, cosmetics, chemicals (e.g., paints, dyes, and colorants), fertilizer and animal feed, and the like. Such methods traditionally include the addition of chemicals or the use of mechanical equipment to physically separate algae from the remaining components of a water slurry. Separation of algal biomass has proven to be a dramatic drain on production costs because harvest-ready algae is typically in low concentrations in a water slurry (e.g., about 1 gram per liter), has low sedimentation velocity, and has a colloidal nature that maintains it in suspension, among other complications. As such, algae does not itself easily settle out of a water slurry and, large volumes of liquid must be processed to concentrate algal biomass, requiring significant pumping and piping costs (i.e., to move such large volumes of water through various processing equipment), energy usage, and material waste.

Because the processing of algal biomass produces valuable commodities, including sustainable biofuels and non-oil based products, cost-effective harvesting methods that overcome some, or all, of the complications traditionally associated with harvesting are desirable. Moreover, it is further desirable that such harvesting methods minimize energy usage, minimize production costs, and minimize material waste.

### SUMMARY OF THE INVENTION

The present disclosure relates to algae harvesting, and more particularly, to integration of algae harvesting and growth systems in a single cultivation vessel, as well as methods related thereto.

In one or more aspects of the disclosure, a system is disclosed that includes a cultivation vessel configured for cultivating an algae water slurry and a harvesting system integrated with the cultivation vessel and configured for selectively harvesting algae cells.

In one or more additional aspects of the disclosure, a method is disclosed that includes containing an algae water slurry in a cultivation vessel, the cultivation vessel having an integrated harvesting system and the algae water slurry comprising algae cells, water, and algae nutrient media. The contained algae water slurry is cultivated for a predetermined period of time and, thereafter, at least a portion of the algae cells are selectively harvested using the integrated harvesting system.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a schematic illustration of a top-view of a standard raceway pond algae cultivation vessel.

FIG. 2A is a schematic illustration of a top-view of a DAF integrated system, in accordance with one or more embodiments of the present disclosure.

FIG. 2B is a schematic illustration of a side-view of the DAF integrated system of FIG. 2A.

FIG. 3A is a schematic illustration of a top-view of a submerged membrane integrated system, in accordance with one or more embodiments of the present disclosure.

FIGS. 3B and 3C are schematic illustrations of a side-view of the submerged membrane integrated system of FIG. 3A during growth and harvesting.

FIG. 4 is a schematic illustration of a top-view of an adjacent membrane integrated system, in accordance with one or more embodiments of the present disclosure.

### DETAILED DESCRIPTION OF THE INVENTION

The present disclosure relates to algae harvesting, and more particularly, to integration of algae harvesting and growth systems in a single cultivation vessel, as well as methods related thereto.

Biofuel production from cultivated algae slurries offers sustainable energy solutions to reduce reliance on fossil fuels and reduce greenhouse gas emissions. Other non-oil-based products can additionally be derived from algal biomass. To accomplish substantial economic, environmental, and societal impact, algae must be cultivated in large-scale systems. Such large-scale cultivation systems allow algae-derived fuels and other non-oil-based products to become more cost-effective and more widely available to the public. However, harvesting algal biomass is traditionally a major bottleneck to large-scale, industrial sized processing in terms of production costs, energy costs, time, and environmental impact, since it requires algal biomass to be effectively separated, or “dewatered,” from the remaining components of extremely large-volume slurries.

Algae and algae cells are cultivated in algae slurries that may be upwards of thousands of liters or more in volume. Algae cells are generally cultivated for a predetermined period of time and allowed to reach maximum concentrations of only about 1000 parts per million (ppm) before the algal biomass is harvested. Accordingly, the amount of algal biomass for harvesting is relatively dilute within the algae slurry. Traditional harvesting methods transport the entire slurry volume from the cultivation vessel to a harvesting facility, or area for separation of the algae biomass, from the remaining components of the slurry. In many systems, this process involves transporting extremely large quantities of slurry water over long distances using pumps, pipes, and other means.

Accordingly, the transportation alone of large-volume slurries to harvesting equipment, and back to cultivation vessels for recycling, incurs significant energy and equipment costs, requiring pumping and piping of sufficient capability and capacity to move the slurries from one location to one or more often quite distant locations. Similarly, separation equipment must be sized and occupy a corresponding facility footprint to effectively harvest and dewater the large-volume slurries. Harvesting of algal biomass is further complicated because algae cells are so dilute in the slurry, and comprise charged surface proteins and other charged compounds, that generally prevent their natural settling within a slurry liquid to facilitate separation.

The present disclosure provides systems and methods of integrated algae harvesting and growth schemes within a single algae cultivation vessel to reduce costs and energy consumption. More particularly, a harvesting unit is integrated with a cultivation vessel, such that after cultivation of an algae slurry for a predetermined period of time, operation of the harvesting unit may transition to selectively harvest or separate a concentrated (e.g., pre-thickened) algae stream from the cultivation vessel and the other water slurry components (e.g., water and nutrients) remain in the cultivation vessel. The volume of the concentrated algae output is greatly reduced compared to the volume of the entire water slurry after cultivation. Accordingly, costs and equipment sizes associated with transporting and further harvesting the concentrated algae output may also be reduced. Moreover, the other water slurry components that remain in the cultivation vessel may be recycled for subsequent algae cultivation purposes without the need to re-transport (recirculate) the media back to the cultivation vessel, or the need to refill an entire new volume of water and nutrients for cultivation. That is, only make-up water and additional algae nutrients may be required to feed the cultivation vessel to begin subsequent algae cultivation (i.e., rather than a full slurry volume) because the remaining water slurry components are already present.

As used herein, the term “algae slurry” or “algae water slurry,” and grammatical variants thereof, refers to a flowable liquid comprising at least water, algae cells, and algae nutrient media (e.g., phosphorous, nitrogen, and optionally additional elemental nutrients).

Algal sources for preparing the algae slurry include, but are not limited to, unicellular and multicellular algae. Examples of such algae can include, but are not limited to, a rhodophyte, chlorophyte, heterokontophyte, tribophyte, glaucophyte, chlorarachniophyte, euglenoid, haptophyte, cryptomonad, dinoflagellum, phytoplankton, and the like, and combinations thereof In one embodiment, algae can be of the classes Chlorophyceae and/or Haptophyta. Specific species can include, but are not limited to, Neochloris oleoabundans, Scenedesmus dimorphus, Euglena gracilis, Phaeodactylum tricornutum, Pleurochrysis carterae, Prymnesium parvum, Tetraselmis chui, and Chlamydomonas reinhardtii. Additional or alternate algal sources can include one or more microalgae of the Achnanthes, Amphiprora, Amphora, Ankistrodesmus, Asteromonas, Boekelovia, Borodinella, Botryococcus, Bracteococcus, Chaetoceros, Carteria, Chlamydomonas, Chlorococcum, Chlorogonium, Chlorella, Chroomonas, Chrysosphaera, Cricosphaera, Crypthecodinium, Cryptomonas, Cyclotella, Dunaliella, Ellipsoidon, Emiliania, Eremosphaera, Ernodesmius, Euglena, Franceia, Fragilaria, Gloeothamnion, Haematococcus, Halocafeteria, Hymenomonas, Isochrysis, Lepocinclis, Micractinium, Monoraphidium, Nannochloris, Nannochloropsis, Navicula, Neochloris, Nephrochloris, Nephroselmis, Nitzschia, Ochromonas, Oedogonium, Oocystis, Ostreococcus, Pavlova, Parachlorella, Pascheria, Phaeodactylum, Phagus, Pichochlorum, Pseudoneochloris, Pseudostaurastrum, Platymonas, Pleurochrysis, Pleurococcus, Prototheca, Pseudochlorella, Pyramimonas, Pyrobotrys, Scenedesmus, Schizochlamydella, Skeletonema, Spyrogyra, Stichococcus, Tetrachlorella, Tetraselmis, Thalassiosira, Tribonema, Vaucheria, Viridiella, and Volvox species, and/or one or more cyanobacteria of the Agmenellum, Anabaena, Anabaenopsis, Anacystis, Aphanizomenon, Arthrospira, Asterocapsa, Borzia, Calothrix, Chamaesiphon, Chlorogloeopsis, Chroococcidiopsis, Chroococcus, Crinalium, Cyanobacterium, Cyanobium, Cyanocystis, Cyanospira, Cyanothece, Cylindrospermopsis, Cylindrospermum, Dactylococcopsis, Dermocarpella, Fischerella, Fremyella, Geitleria, Geitlerinema, Gloeobacter, Gloeocapsa, Gloeothece, Halospirulina, Iyengariella, Leptolyngbya, Limnothrix, Lyngbya, Microcoleus, Microcystis, Myxosarcina, Nodularia, Nostoc, Nostochopsis, Oscillatoria, Phormidium, Planktothrix, Pleurocapsa, Prochlorococcus, Prochloron, Prochlorothrix, Pseudanabaena, Rivularia, Schizothrix, Scytonema, Spirulina, Stanieria, Starria, Stigonema, Symploca, Synechococcus, Synechocystis, Tolypothrix, Trichodesmium, Tychonema, and Xenococcus species.

The water for use in preparing the algae slurry may be from any water source including, but not limited to, fresh water, brackish water, seawater, wastewater (treated or untreated), synthetic seawater, and any combination thereof.

The algae nutrient media for use in forming an algae slurry may comprise at least nitrogen (e.g., in the form of ammonium nitrate or ammonium urea) and phosphorous. Other elemental micronutrients may also be included, such as potassium, iron, manganese, copper, zinc, molybdenum, vanadium, boron, chloride, cobalt, silicon, and the like, and any combination thereof.

As used herein, the term “cultivation vessel,” “vessel,” and grammatical variants thereof, refers to any of an open or closed algae cultivation system used for the growth of algal biomass, including bioreactors, photobioreactors, natural ponds, artificial ponds (e.g., raceway ponds), and the like. While the embodiments of the present disclosure are generally described with reference to open cultivation systems, such as natural or artificial ponds, it is to be appreciated that either open or closed cultivation systems may be used in accordance with the embodiments of the present disclosure.

As used herein, the term “growth system” or “growth scheme,” and grammatical variants thereof, includes the particular mixing means and algae slurry utilized to grow a particular type of algae contained within a cultivation vessel, and is not considered to be particularly limited.

As used herein, the term “harvesting unit” or “harvesting system,” and grammatical variants thereof, refers to any equipment or equipment system that can be integrated with a cultivation vessel and used to selectively harvest (separate) algae and algae cells from an algae slurry. Generally, the harvesting units described herein comprise one or more components to achieve desired harvesting of a concentrated algal biomass. Examples of suitable harvesting unit components may include, but are not limited to, a skimmer (e.g., a weir skimmer), a membrane filter (or membrane module comprising one or more membranes), an air sparger, a pump, one or more conduits (e.g., pipes, hoses, channels, troughs, and the like), suitable valving, and the like, and any combination thereof. Combinations of particular components to form specific harvesting units are described hereinbelow.

As used herein, the term “integrated,” and grammatical variants thereof, with reference to a harvesting unit and cultivation vessel means that the unit and vessel are co-located and operate or function as a single assembly. For example, at least one component of the harvesting unit may be in fluid communication with the cultivation vessel. For ease of reference, a single integrated harvesting unit and cultivation vessel will be described herein simply as an “integrated system.”

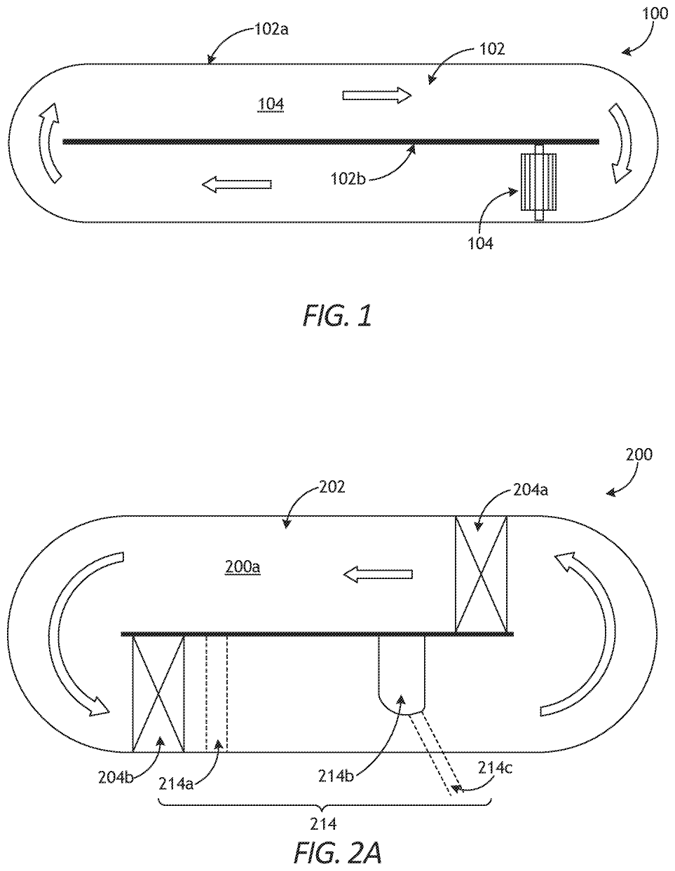

For purposes of the present disclosure, the integrated system embodiments herein will be described with reference to open cultivation systems and, more particularly, with reference to open cultivation raceway ponds. Accordingly, before describing various specific integrated system embodiments in detail, a brief overview of standard open algae cultivation raceway ponds that may be utilized in accordance with one or more embodiments of the present disclosure is provided with reference to FIG. 1. As illustrated in FIG. 1, raceway pond 100 is a single closed-loop, oval-shaped (e.g., pill-shaped) pond having recirculation channel 102. Raceway pond 100 may be artificial and shallow, designed to circulate an algae slurry at a depth of no greater than about 12 inches to facilitate sufficient sunlight penetration needed for algae growth.

Although FIG. 1 shows raceway pond 100 having a single closed-loop, oval-shaped recirculation channel 102, in other embodiments, a suitable raceway pond may have multiple closed-loop, oval-shaped recirculation channels, or may comprise other shaped recirculation channels, without departing from the scope of the disclosure. A circulation device 104 may be located at one or more locations within the channel 102 to enable circulation (e.g., shown as arrows in FIG. 1) and prevent sedimentation of the contents of an algae water slurry cultivated within the channel 102. Typically, circulation device(s) 104 are located before one or more curved sections of raceway pond 100, but may be positioned at other locations provided that sufficient bulk liquid flow velocity is maintained. Examples of suitable circulation device(s) 104 include powered mechanical devices, such as paddlewheels, jet mixers, airlift pumps, mixing boards, and the like, which may be used singly or in combination within channel 102 of raceway pond 100.

The integrated systems of the present disclosure may have one or more components of a harvesting unit integrated with a cultivation vessel, and the cultivation vessel may be identical, similar, or different from raceway pond 100 of FIG. 1. The components of a harvesting unit may be integrated with the cultivation vessel, for example, such as by submersion within channel 102, fixation to one or more walls 102a,b of channel 102, otherwise in fluid communication with one or more other components of the cultivation vessel, and any combination thereof.

In some embodiments, the integrated systems of the present disclosure may include a type of dissolved air floatation (DAF) harvesting unit (a “DAF integrated system”), or a type of membrane harvesting unit(s) (a “membrane integrated system”). In other embodiments, the integrated systems may include a combination of both of one or more DAF harvesting units and one or more membrane harvesting units.

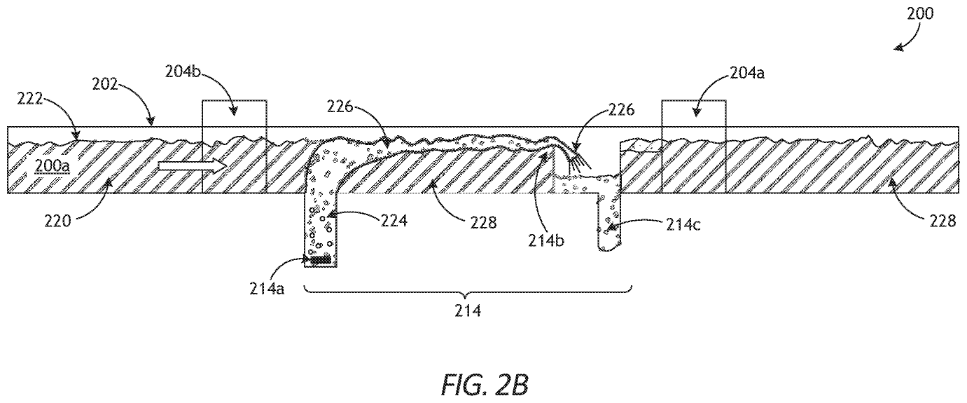

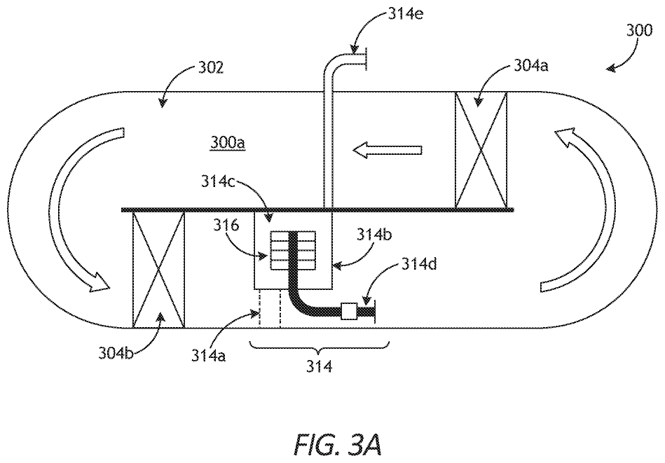

FIG. 2A illustrates a top-view of a DAF integrated system 200, in accordance with one or more embodiments of the present disclosure. The DAF integrated system 200 of FIG. 2A comprises a cultivation vessel 200a having, as shown, two circulation devices 204a,b. The circulation devices 204a,b may be any device (e.g., powered mechanical device) operable to cause circulation of an algae slurry contained within recirculation channel 202, such as those described hereinabove. It is further to be appreciated that the cultivation vessel 200a may include solely one or greater than two circulation devices 204a,b, which may be located at any other location along the cultivation vessel 200a, without departing from the scope of the present disclosure. The flow of the algae slurry follows the unlabeled arrows shown within the recirculation channel 202, the direction of flow being non-limited (e.g., may be clockwise or counterclockwise).

The DAF integrated system 200 may include a DAF harvesting system 214, which may include an air sparger 214a, a skimmer 214b, and an algae output conduit 214c, which are collectively integrated with the cultivation vessel 200a. The air sparger 214a functions to inject pressurized gas (e.g., air, CO2, and the like) into the circulating algae slurry in the recirculation channel 202 to feed the algae and lift cultivated algal biomass to the surface of the slurry. The skimmer 214b functions to skim the biomass that was lifted by the air sparger 214a from the surface of the slurry in the channel 202 as it flows past the skimmer 214b. Finally, the algae output conduit 214c functions as an outlet or discharge passage through which the skimmed biomass may be removed from the channel 202, such as to another container for further dewatering and processing. Accordingly, the DAF integrated system 200 may continuously remove concentrated cultivated biomass as the biomass encounters the DAF harvesting system 214, while ensuring that the remaining components of the slurry are contained within the channel 202. Such a system may substantially decrease costs, energy, and time related to traditional harvesting methods, requiring handling and transportation of large volumes of water slurries, as described hereinabove.

Referring now to FIG. 2B, illustrated is a more-detailed, side-view of the DAF integrated system 200 of FIG. 2A. Accordingly, like labels from FIG. 2A will be used with reference to FIG. 2B. DAF integrated system 200 comprises cultivation vessel 200a, recirculation channel 202, and circulation devices 204a,b. The flow of the algae slurry follows the unlabeled arrow within channel 202. DAF harvesting system 214 comprises air sparger 214a, skimmer 214b (shown as a weir), and algae output conduit 214c. As shown, air sparger 214a is positioned in a sump; however, it is to be noted that air sparger 214a may be located within the channel 202 (e.g., on a wall or the bottom surface) or otherwise suspended within channel 202, without departing from the scope of the present disclosure.

With continued reference to FIG. 2B, the operation of the DAF integrated system 200 is now described. Algae water slurry 220 circulates within the channel 202 following the direction of the unlabeled arrow and has a surface 222. After the algae water slurry 220 has been cultivated for a predetermined period of time, algal biomass is ready for harvesting. At this stage, the operation of the integrated DAF harvesting system 214 may commence. Air sparger 214a creates bubbles 224 within the slurry 220, which comprises the cultivated biomass. The generated air bubbles 224 penetrate the slurry 220 and float the cultivated biomass, such that a floated mixture 226 of bubbles and concentrated cultivated biomass rise to the surface 222. The remaining components 228 of the slurry 220 (e.g., water and nutrients) are located below the floated mixture 226, and may comprise a clarified water mixture. Thereafter, the floated mixture 226 encounters the skimmer 214b (weir), which causes the floated mixture 226 comprising the concentrated biomass to be directed or otherwise collected by the algae output conduit 214c, while the remaining components 228 continue to be circulated within the channel 202 (e.g., past circulation device 204b).

As provided above, another type of integrated system may utilize a membrane harvesting system, alone or in combination with a DAF harvesting system. The membrane harvesting system may be integrated with a cultivation vessel in non-limiting configurations. In some embodiments, for example, the membrane system may be submerged within or adjacent to a cultivation vessel, wherein an algae slurry comprising cultivated biomass may be pumped over or through one or more membranes to concentrate the biomass and return the remaining components of an algae slurry to the cultivation vessel of the membrane(s).

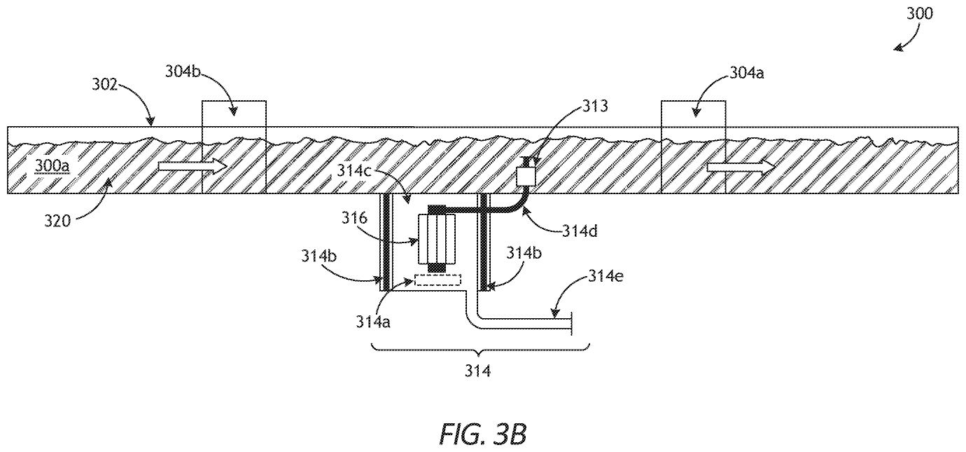

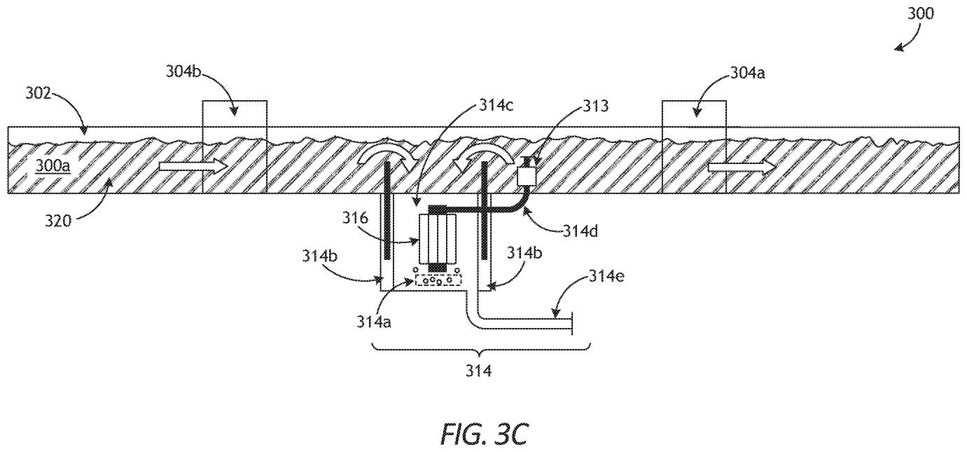

FIG. 3A illustrates a top-view of an example of a submerged membrane integrated system 300, in accordance with one or more embodiments of the present disclosure. The membrane integrated system 300 comprises a cultivation vessel 300a, including two circulation devices 304a,b, which may be any device (e.g., powered mechanical device) to cause circulation of an algae slurry contained within recirculation channel 302, such as those described hereinabove. Similar to FIGS. 2A and 2B, one or more of the circulation devices 304a,b may be located at any other location along the recirculation channel 302, without departing from the scope of the present disclosure. The flow of the algae slurry follows the unlabeled arrows shown within the recirculation channel 302, the direction of flow being non-limited.

The membrane integrated system 300 may include a submerged membrane harvesting system 314, which may include an air sparger 314a, a skimmer 314b, a membrane module 314c, a permeate conduit 314d (shown having pump 313 in FIGS. 3B and 3C to facilitate flow), and an algae output conduit 314e, which are collectively integrated with cultivation vessel 300a. The air sparger 314a and skimmer 314b function to direct the circulating algae slurry to the membrane module 314c. More specifically, in some embodiments, the skimmer 314b may be an adjustable skimmer (e.g., adjustable weir) that moves vertically, within and below the bottom of channel 302, to control the amount of water that may access the membrane module 314c, and to permit free-flow during cultivation, as described in greater detail hereinbelow. In concert, the air sparger 314a functions to inject pressurized gas into the algae slurry that flows over the skimmer 314b toward the membrane module 314c.

The membrane module 314c may include one or more membrane arrangements to filter cultivated algal biomass from the algae slurry. In non-limiting examples, the membrane module 314c may contain one or more cassettes comprising one or more fiber membranes, which may be of any shape (e.g., tubular, planar, spiral, etc.). The fiber membranes comprise a semi-permeable barrier. Components of a fluid flowing past the fiber membranes that are permeable to the semi-permeable barrier flow through the fiber membranes as permeate, whereas components of a fluid flowing past the fiber membranes that are not permeable to the semi-permeable membrane are retained by the membrane (e.g., removably adhered to the membrane). In some instances, the fiber membranes may be in the form of a hollow fiber membrane, wherein components of a fluid that are permeable to the semi-permeable barrier are retained within an interior channel and can exit the interior channel through one or more ports.

The fiber membrane(s) may be composed of any suitable materials, typically one or more polymers and/or ceramics. In some embodiments, the fiber membrane(s) of the present disclosure may be composed of a ceramic, polyvinylidene fluoride, polyvinylidene difluoride, polytetrafluoroethylene, a polyether sulfone, cellulose (e.g., cellulose triacetate), a polyamide, polyacrylonitrile, and the like, and any combination thereof.

Accordingly, and with continued reference to FIG. 3A, the membrane(s) 316 may be in the form of a hollow fiber membrane(s). An algae slurry may be flowed outside of the membrane(s) 316 in the membrane module 314c and the membrane(s) 316 may retain and concentrate the cultivated algal biomass, whereas the remaining water and nutrients (permeate) may flow into the hollow interior of the membrane 316. The permeate may be pumped or otherwise flow out of the hollow interior of the membrane(s) 316 (e.g., through a port) in the membrane module 314c through permeate conduit 314d and returned to the channel 302. The retained, concentrated algal biomass may be removed from the membrane surface and collected through algae output conduit 314e for further dewatering and processing. The algal biomass may be removed from the membrane surface using any non-limiting method, such as, for example, by air scouring, liquid flushing, and any combination thereof For example, air scouring may be used to blow off material (e.g., algal biomass) captured by the membrane surface and/or forward/backward liquid flushing may be used to push off material (e.g., algal biomass) captured by the membrane surface. Accordingly, the submerged membrane integrated system 300 may continuously remove concentrated algal biomass from the circulating algae slurry, while retaining the remaining components of the slurry within the channel 302, thereby reducing costs, energy, and time.

Referring now to FIGS. 3B and 3C, illustrated are side-views of the submerged membrane system 300 of FIG. 3A. Accordingly, like labels from FIG. 3A will be used with reference to FIGS. 3B and 3C. As shown in FIGS. 3B and 3C, illustrated is submerged membrane integrated system 300 comprising cultivation vessel 300a, recirculation channel 302, and circulation devices 304a,b. Integrated with the cultivation vessel 300a is submerged harvesting system 314 (e.g., located within a sump beneath the cultivation vessel 300a bottom) comprising air sparger 314a, skimmer 314b, membrane module 314c, permeate conduit 314d (and pump 313), and algae output conduit 314e.

The growth or cultivation phase of algae slurry 320 is shown in FIG. 3B. More particularly, the skimmer 314b is adjustable (e.g., retractable) and is retracted at least partially, or wholly, below the bottom of the cultivation vessel 300a, thereby permitting free-flow circulation of the algae slurry 320. With the skimmer(s) 314b retracted, the algae slurry 320 flows in the direction of the unlabeled arrows within channel 302 without substantial hindrance from the skimmer 314b.

Referring now to FIG. 3C, the harvesting phase of algae slurry 320 is shown. The adjustable skimmer 314b is advanced (extended) into the channel 302 of cultivation vessel 300a. The skimmer 314b is advanced into the channel 302 no further than the surface of the circulating algae slurry 320, such that at least a portion of the algae slurry 320 is able to flow toward the membrane module 314c; the remainder of the algae slurry 320 may be bypassed and continue to be circulated in the channel 302. The particular distance that the skimmer 314b is advanced (extended) into the channel 302, which correlates to the distance between the surface of the algae slurry 320 and the top of the skimmer 314b, may be used to control the amount of slurry that is directed toward the membrane module 314c.

Air sparger 314a may be present to ensure that the membrane surface remains substantially clean and free from fouling. Further, the bubbles created by the air sparger 314a may to assist in flowing the directed algae slurry 320 over the one or more membranes 316. Cultivated algae biomass is retained and concentrated on membrane(s) 316 and the remaining algae slurry 320 components (e.g., water and nutrients) permeate through the membrane(s) 316 and into the hollow interior thereof for collection by the permeate conduit 314d. The collected components of the algae slurry 320 by the permeate conduit 314d are returned to the channel 302 of the cultivation vessel 300a, which may be aided using pump 313, for reuse in subsequent algae cultivation activities. The retained and concentrated algal biomass is removed and collected through algae output conduit 314e by one or more removal methods, such as those described hereinabove.

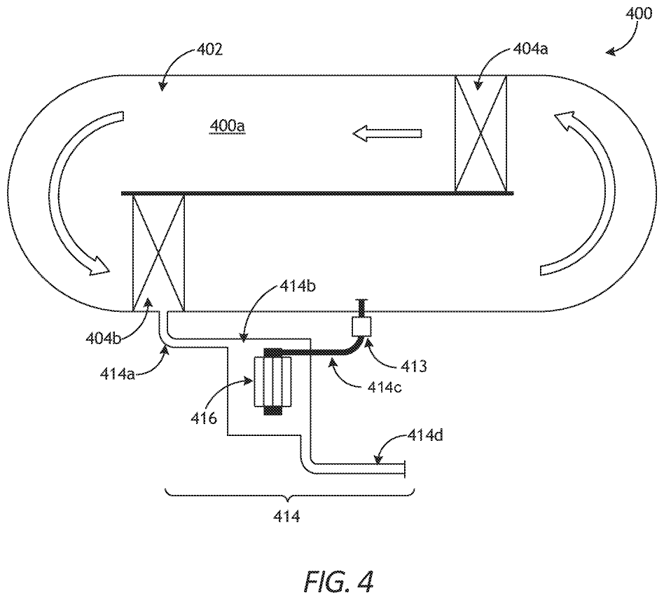

Alternative to a submerged membrane integrated system, the present disclosure further provides for a membrane integrated system configuration in which a membrane harvesting unit is adjacent to a cultivation vessel and otherwise fluidly coupled thereto. FIG. 4 illustrates a top-view of an adjacent membrane integrated system 400, in accordance with one or more embodiments of the present disclosure. The components of adjacent membrane integrated system 400 may be the same or similar to those described in FIGS. 3A-3C, and thus operate in the same or similar fashion to accomplish the same or similar results, unless specified otherwise. As shown in FIG. 4, the membrane integrated system 400 may include a cultivation vessel 400a having, as shown, two circulation devices 404a,b, which may be any device (e.g., powered mechanical device) to cause circulation of an algae slurry contained within recirculation channel 402, as described hereinabove. Similar to FIGS. 2A-3C, one or greater than two circulation devices may be located at any other location along the cultivation vessel 400a, without departing from the scope of the present disclosure. The flow of the algae slurry follows the unlabeled arrows shown within the recirculation channel 402, the direction of flow being non-limited.

The membrane integrated system 400 comprises an adjacent membrane harvesting system 414, which may include a slipstream conduit 414a, a membrane module 414b (having one or more membranes 416, such as a fiber membrane), a permeate conduit 414c (shown having pump 413 to facilitate flow), and an algae output conduit 414d. The slipstream conduit 414a may utilize the current generated by the circulation device 404b to direct at least a portion of the circulating algae slurry to the membrane module 414b. It is to be noted, however, that an additional one or more pumps, gas spargers, etc. may be used to further facilitate flow into the slipstream conduit 414a. Alternatively, the slipstream conduit 414a may be located at a location that does not rely (or only minimally relies) on a slipstream from a circulation device (e.g., located upstream of a circulation device), and one or more pumps may be used in such instances to facilitate flow (which may be referred to as a slurry input conduit), without departing from the scope of the present disclosure.

In the illustrated embodiment, the algae slurry flows over the membrane(s) 416 in membrane module 414b and the membrane(s) 416 may retain and concentrate cultivated algal biomass, whereas the remaining water and nutrients (permeate) flow through and outside of the membrane(s) 416 into the membrane module 414b. The permeate may be pumped or otherwise flow out of the membrane module 414b through permeate conduit 414c and returned (recycled) to the channel 402. The retained, concentrated algal biomass may be removed from the membrane surface and collected through algae output conduit 414d for further dewatering and processing, such as by one or more removal methods described hereinabove. Accordingly, the adjacent membrane integrated system 400 may continuously remove concentrated algal biomass from the circulating algae slurry, while retaining the remaining components of the slurry within the channel 402, thereby reducing costs, energy, and time.

### Embodiments Listing

The present disclosure provides, among others, the following embodiments, each of which may be considered as optionally including any alternate embodiments.

Clause 1. A system comprising: a cultivation vessel configured for cultivating an algae water slurry; and a harvesting system integrated with the cultivation vessel and configured for selectively harvesting algae from the algae water slurry.

Clause 2. The system of Clause 1, wherein the cultivation vessel defines a reticulation channel and the integrated system is in fluid communication with the recirculation channel.

Clause 3. The system of Clause 3, further comprising at least one circulation device arranged within the recirculation channel to circulate the algae water slurry.

Clause 4. The system of any of Clauses 1 to 3, wherein the harvesting system is a dissolved air flotation harvesting system.

Clause 5. The system of Clause 4, wherein the dissolved air floatation harvesting system comprises: an air sparger; a skimmer; and an algae output conduit.

Clause 6. The system of Clause 5, wherein the skimmer is a weir.

Clause 7. The System of any of Clauses 1 to 3, wherein the harvesting system is a membrane harvesting system.

Clause 8. The system of Clause 7, wherein the membrane harvesting system comprises: a membrane module having one or more membranes; a permeate conduit; and an algae output conduit.

Clause 9. The system of any of Clauses 7 or 8, wherein the cultivation vessel defines a recirculation channel and the membrane harvesting system is submerged within the recirculation channel.

Clause 10. The system of any of Clauses 7 or 8, wherein the membrane harvesting system is located adjacent the cultivation vessel and in fluid communication with the cultivation vessel via an input conduit.

Clause 11. The system of any of Clauses 7 to 10, wherein the membrane harvesting system further comprises an air sparger and a skimmer.

Clause 12. The system of any of Clauses 11, wherein the skimmer is an adjustable skimmer.

Clause 13. The system of any of Clauses 1 to 12, wherein the cultivation vessel is a raceway pond.

Clause 14. A method, comprising: containing within a cultivation vessel an algae water slurry comprising algae cells, water, and algae nutrient media; cultivating the algae water slurry for a predetermined period of time; and selectively harvesting at least a portion of the algae cells from the algae water slurry using a harvesting system integrated into the cultivation vessel.

Clause 15. The method of Clause 14, wherein the cultivation vessel includes a recirculation channel, the method further comprising establishing fluid communication between the harvesting system and the recirculation channel.

Clause 16. The method of any of Clauses 14 or 15, wherein the harvesting system is a dissolved air floatation harvesting system, and wherein selectively harvesting the algae cells from the algae water slurry comprises collecting floated algae cells in the algae water slurry.

Clause 17. The method of any of Clauses 14 or 15, wherein the harvesting system is a membrane floatation harvesting system, and wherein selectively harvesting the algae cells from the algae water slurry comprises collecting membrane-adhered algae cells from the algae water slurry.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art, having the benefit of the teachings herein. Furthermore, no limitations are intended to the details of construction or design herein shown, other than as described in the claims below. It is, therefore, evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

What is claimed is:

1. A system comprising:
a cultivation vessel configured for cultivating an algae water slurry; and
a harvesting system integrated with the cultivation vessel and configured for selectively harvesting algae from the algae water slurry.

2. The system of claim 1, wherein the cultivation vessel defines a recirculation channel and the integrated harvesting system is in fluid communication with the recirculation channel.

3. The system of claim 2, further comprising at least one circulation device arranged within the recirculation channel to circulate the algae water slurry.

4. The system of claim 1, wherein the harvesting system is a dissolved air flotation harvesting system.

5. The system of claim 4, wherein the dissolved air floatation harvesting system comprises:
an air sparger;
a skimmer; and
an algae output conduit.

6. The system of claim 5, wherein the skimmer is a weir.

7. The system of claim 1, wherein the harvesting system is a membrane harvesting system.

8. The system of claim 7, wherein the membrane harvesting system comprises:
a membrane module having one or more membranes;
a permeate conduit; and
an algae output conduit.

9. The system of claim 8, wherein the cultivation vessel defines a recirculation channel and the membrane harvesting system is submerged within the recirculation channel.

10. The system of claim 9, wherein the membrane harvesting system further comprises an air sparger and a skimmer.

11. The system of claim 10, wherein the skimmer is an adjustable skimmer.

12. The system of claim 8, wherein the membrane harvesting system is located adjacent the cultivation vessel and in fluid communication with the cultivation vessel via an input conduit.

13. The system of claim 1, wherein the cultivation vessel is a raceway pond.

14. A method, comprising:
containing within a cultivation vessel an algae water slurry comprising algae cells, water, and algae nutrient media;
cultivating the algae water slurry for a predetermined period of time; and
selectively harvesting at least a portion of the algae cells from the algae water slurry using a harvesting system integrated into the cultivation vessel.

15. The method of claim 14, wherein the cultivation vessel includes a recirculation channel, the method further comprising establishing fluid communication between the harvesting system and the recirculation channel.

16. The method of claim 14, wherein the harvesting system is a dissolved air floatation harvesting system, and wherein selectively harvesting the algae cells from the algae water slurry comprises collecting floated algae cells in the algae water slurry.

17. The method of claim 14, wherein the harvesting system is a membrane floatation harvesting system, and wherein selectively harvesting the algae cells from the algae water slurry comprises collecting membrane-adhered algae cells from the algae water slurry.
